(19) United States
(12) Patent Application Publication
(10) Pub. No.: US 2026/0325416 A1
(43) Pub. Date: Oct. 22, 2026

(54) POWDER COSMETIC PREPARATION

(71) Applicant: SHISEIDO COMPANY, LTD., Chuo-ku, Tokyo (JP)

(72) Inventors: Tatsuya YONEMIZU, Tokyo (JP); Yui YAMAGUCHI, Tokyo (JP)

(73) Assignee: SHISEIDO COMPANY, LTD., Chuo-ku, Tokyo (JP)

(21) Appl. No.: 19/110,497

(22) PCT Filed: Sep. 29, 2023
(86) PCT No.: PCT/JP2023/035575

§ 371 Date: Mar. 11, 2025

(30) Foreign Application Priority Data
Oct. 13, 2022 (JP) 2022-164627

(51) Int. Cl. A61K 8/25; A61K 8/02; A61K 8/29; A61K 8/36; A61Q 1/12; A61Q 17/04

(52) U.S. Cl. CPC A61K 8/25; A61K 8/022; A61K 8/0241; A61K 8/29; A61K 8/361; A61Q 1/12; A61Q 17/04; A61K 2800/412; A61K 2800/413; A61K 2800/612

(57) ABSTRACT

The purpose of the present invention is to provide a powder cosmetic preparation which achieves a high ultraviolet light shielding performance without causing aggregation of fine particles of a metal oxide powder (such as titanium oxide powder) that serves as an ultraviolet scattering agent even if a large amount of the metal oxide powder is added thereto, and which has excellent texture. The present invention relates to a powder cosmetic preparation which is characterized by containing (a) at least one metal oxide powder that is selected from among titanium oxide, zinc oxide and cerium oxide and has an average particle diameter of 10 nm or less in an amount of 5% by mass or more relative to the total amount of the cosmetic preparation, and (b) silica. It is preferable that titanium oxide is used as the metal oxide powder (a).

### TECHNICAL FIELD

The present invention relates to powder cosmetic preparations (or “powder cosmetics”). More specifically, the present invention relates to powder cosmetics which have a high UV protection ability based on a large amount of particulate metal oxide powder (which is a UV scattering agent) but in which no aggregation of the particulate metal oxide powder occurs.

### BACKGROUND ART

Face powder is a kind of makeup cosmetic that has been widely used for a long time; however, its role has changed with the times, and its main purpose has been to eliminate the greasy shine caused by sweat and sebum and to improve makeup retention. Today, however, as it has become common to wear masks to prevent the spread of infectious diseases such as COVID-19, cosmetic behaviors tend to be simplified. As a result, more consumers are demanding high performance, such as high SPF (Sun Protection Factor), for powder cosmetics, such as face powder, that are easy to apply.

The improvement of the UV protection ability of powder cosmetics such as face powders requires increasing the amounts of UV absorbing agents or UV scattering agents. However, since the UV absorbing agents are oily substances, it is difficult to formulate it in loose powder cosmetics (such as loose face powder) that contain almost no oil. In addition, high contents of oily substances such as UV absorbing agents may induce aggregation of powder and impair the uniformity and texture of the cosmetics.

Metal oxide powder, such as particulate titanium oxide, zinc oxide, or cerium oxide, are commonly used as UV scattering agents. For example, a cosmetic containing fine particulate metal oxide having an average particle size of 300 Å (30 nm) or less exhibits a UV shielding effect and is also excellent in transparency (Patent Document 1).

On the other hand, it is also known that when the particle size of the powder becomes smaller, powder aggregation is more likely to occur. Thus, even when a large amount of particulate titanium oxide or the like is contained, the improvement in the UV protection ability (high SPF) expected from the increase in the amount has not been obtained due to the aggregation of powder. In addition, the high content of metal oxide powder (such as a UV scattering agent) may cause squeakiness which results in degradation of texture upon application.

That is, suppressing the aggregation of the powder and solving the texture problem are required to achieve a high UV protection ability based on the particulate metal oxide.

### CITATION LIST

### Patent Document

Patent Document 1: JP-A Sho 62-84017

### SUMMARY OF INVENTION

### Technical Problem

An object of the present invention is to provide a powder cosmetic having a high UV protection ability without aggregation even when a large amount of a particulate metal oxide powder (such as titanium oxide) is contained as a UV scattering agent, and further having an excellent texture.

### Solution to Problem

As a result of intensive studies to solve the above problems, the present inventors have found that the aggregation of the metal oxide powder is suppressed and a high UV protection ability is achieved by containing silica, especially spherical silica, while increasing the amount of a particulate metal oxide powder which is a UV scattering agent, and have completed the present invention.

That is, the present invention provides a powder cosmetic, comprising: (a) metal oxide powder consisting of at least one selected from titanium oxide, zinc oxide, and cerium oxide, and having an average particle size of 10 nm or less, in an amount of 5% by mass or more with respect to a total amount of the cosmetic; and (b) silica.

### Advantageous Effects of Invention

The powder cosmetic of the present invention contains metal oxide powder selected from titanium oxide powder, zinc oxide powder and cerium oxide powder, and has an average particle size of 10 nm or less in an amount of 5% by mass or more. The powder cosmetic of the present invention exerts a high UV protection ability based on the metal oxide powder, i.e., a UV scattering agent, contained in an amount of 5% by mass or more, but no powder aggregation occurs even when such a large amount of metal oxide powder is contained. It is recognized that the co-formulation of the (b) silica with the UV scattering agent in the powder cosmetic of the present invention can provide the improvement of the UV-protection ability based only on the UV scattering agent (hereinafter, this improving effect is also referred to as “boosting effect”). This boosting effect is particularly significant when a particulate powder (metal oxide powder) having an average particle size of 10 nm or less is used as the UV scattering agent.

### BRIEF DESCRIPTION OF DRAWING

FIG. 1 is a graph showing the absorbance in the UV wavelength region of the cosmetics of Comparative Example 1 and Examples 1 to 3 shown in Table 1. The FIGURE shows absorbance curves of (a) Comparative Example 1, (b) Example 1, (c) Example 2, and (d) Example 3.

### DESCRIPTION OF EMBODIMENTS

The powder cosmetic of the present invention (hereinafter also referred to simply as “cosmetic”) comprises (a1) titanium oxide or (a2) zinc oxide and/or (a3) cerium oxide, and further comprises (b) silica.

### (a) Metal Oxide Powder

The “metal oxide powder” (also referred to as “component (a)”) in the cosmetic of the present invention refers to a powder consisting of at least one selected from (a1) titanium oxide (TiO₂, titanium (IV) oxide or titanium dioxide), (a2) zinc oxide (ZnO), and (a3) cerium oxide (CeO2, Cerium (VI) oxide or cerium dioxide).

The “(a) metal oxide powder” in the present invention is a particulate powder having an average primary particle size of 10 nm or less, preferably less than 10 nm.

The average primary particle size means the arithmetic average value of the primary particle size of the powder as measured by commonly used methods. As used herein, the feret diameter of the powder particles measured by the microscopy method is taken as the primary particle size of the particles, and the arithmetic average value calculated from the primary particle size distribution (particle size distribution) determined for the plurality of particles is taken as the average primary particle size (or average particle size). The feret diameter is the distance between two parallel lines when the projected image of the particle is sandwiched by the two parallel lines in a certain direction.

The lower limit of the average particle size of the “(a) metal oxide powder” is not particularly limited, and is usually 1 nm or more, preferably 5 nm or more.

The particle form of the “(a) metal oxide powder” is not particularly limited, and examples thereof include spherical, elliptical, and crushed forms.

Unless otherwise noted herein, the “(a) metal oxide powder” means “(a) a powder of (a1) titanium oxide, (a2) zinc oxide, or (a3) cerium oxide, having an average particle size of 10 nm or less (or less than 10 nm)”.

In the present invention, it is preferable to use (a1) titanium oxide as the “(a) metal oxide powder”.

Powder cosmetics may contain powders having various average particle sizes. For example, titanium oxide, zinc oxide, and the like having an average particle size of about 200 nm or less are generally called as “UV scattering agent”, but when the average particle size thereof is not 10 nm or less, they do not fall under the “component (a)” of the present invention. In addition, metal oxide powders having an average particle size of more than about 200 nm are generally called as “pigment-grade metal oxide powder” and are of course not included in the “component (a)” of the present invention.

In the cosmetic of the present invention, a powder surface-treated with “metallic soap” may be used as the (a) metal oxide powder. The use of the metal oxide powder surface-treated with metallic soap is preferable because the dispersibility of the powder is further improved.

The “metallic soap” is a salt of a saturated or unsaturated higher fatty acid with a metal (other than alkali metal). The higher fatty acid that constitutes the metallic soap is not particularly limited, but examples thereof include saturated and/or unsaturated higher fatty acids having 8 to 24 carbon atoms, particularly 12 to 18 carbon atoms, such as stearic acid, isostearic acid, myristic acid, and lauric acid. The metal constituting the metallic soap is preferably aluminum, calcium, magnesium, zinc, or the like.

Specific examples of the metallic soap preferably used as the surface treatment agent for the (a) metal oxide powder include aluminum stearate, aluminum myristate, magnesium myristate, zinc stearate, calcium stearate, zinc myristate, zinc oleate, magnesium isostearate, magnesium stearate, aluminum distearate, aluminum oleate, aluminum palmitate, aluminum laurate, and aluminum dimyristate.

The “metal oxide powder treated with metallic soap” in the present invention is a powder in which a metal oxide powder to be a base (mother core) is surface-treated with a treatment agent containing a component constituting the metallic soap.

The surface treatment with metallic soap may be performed by a method of surface-treating with a pre-formed metallic soap. For example, the surface treatment can be performed by dissolving metallic soap in a volatile solvent such as isoparaffin, isopropyl alcohol, or the like, mixing it with a base powder, and then volatilizing the volatile solvent. The surface treatment can also be performed by simply mixing a base powder with metallic soap. Alternatively, the surface treatment may be performed by a method of compounding a higher fatty acid (e.g., stearic acid) that constitutes metallic soap with a hydroxide of metal (e.g., aluminum). The surface treatment method is not particularly limited, but examples thereof include a wet method using a solvent, a gas phase method, and a mechanochemical method.

The content of the (a) metal oxide powder in the cosmetic of the present invention is 5% by mass or more, preferably 10% by mass or more, more preferably 20% by mass or more with respect to the total amount of the cosmetic. When the content is less than 5% by mass, sufficient UV protection ability cannot be obtained. The upper limit of the content of the (a) metal oxide powder is not particularly limited, and is usually 40% by mass or less, preferably 30% by mass or less. The range of the content of the (a) metal oxide powder can be, for example, 5 to 40% by mass, 5 to 30% by mass, 10 to 40% by mass, 10 to 30% by mass, 20 to 40% by mass, or 20 to 30% by mass.

### (b) Silica

The “silica (anhydrous silicic acid)” (also referred to as “component (b)”) in the powder cosmetic of the present invention is preferably “spherical silica”. When the spherical silica is used, the “squeakiness” caused by the powder component can be mitigated and the texture of the cosmetic improves. The “spherical” includes those having a shape of true sphere or a substantially spherical shape in which the ratio of major/minor diameters of particles is 1.5 or less, preferably 1.2 or less. By incorporating the silica, the powder cosmetic of the present invention can have an effect improving UV protection ability (“boosting effect”).

The silica (component (b)) is a spherical powder having, but not particularly limited to, an average particle size of 1 to 30 μm, preferably 1 to 20 μm, more preferably 2 to 15 μm. The average particle size of silica is the arithmetic average value of particle size measured according to the laser diffraction and scattering method.

The silica (component (b)) may be non-porous or porous. It is preferred, in particular, to use spherical silica with a specific surface area of 160 m²/g or more, preferably 300 m²/g or more, more preferably 500 m²/g or more, to obtain a good boosting effect.

The specific surface area of silica can be calculated, for example, by measuring the amount of nitrogen adsorbed to the powder at 77 K and analyzing it by the BET method.

The “silica (component (b))” used in the present invention is preferably untreated silica, particularly an untreated spherical silica powder, which has not been surface-treated with a treatment agent including metallic soap or the like.

The content of the (b) silica in the cosmetic of the present invention is preferably 10% by mass or more, more preferably 20% by mass or more, further preferably 30% by mass or more, with respect to the total amount of the cosmetic. The upper limit of the content of the (b) silica is not particularly limited, and is usually 60% by mass or less, preferably 50% by mass or less, with respect to the total amount of the cosmetic. The range of the content of the (b) silica can be, for example, 10 to 60% by mass, 10 to 50% by mass, 20 to 60% by mass, 20 to 50% by mass, 30 to 60% by mass, or 30 to 50% by mass.

### (c) Extender Pigment

Powder cosmetics often contain an inorganic pigment called an “extender pigment”, to maintain the dosage form. The cosmetic of the present invention can also contain the “extender pigment” (also referred to as a component (c)). Specific examples of the “(c) extender pigment” include powders of talc, mica, sericite, kaolin, calcium carbonate, magnesium carbonate, aluminum oxide, barium sulfate, and the like.

The (c) extender pigment in the cosmetic of the present invention can be selected from an untreated extender pigment that has not been surface-treated, or an extender pigment that has been surface-treated with metallic soap, a silicone-based or amino acid-based treatment agent, or the like. However, the content of the extender pigment surface-treated with metallic soap is preferably less than 25% by mass, more preferably less than 20% by mass. The present invention encompasses a cosmetic which does not contain an extender pigment surface-treated with metallic soap.

The cosmetic of the present invention is a powder cosmetic containing (a) a metal oxide powder having an average particle size of 10 nm or less, and (b) silica, and optionally (c) an extender pigment, and the powder cosmetic may further contain other optional components conventionally used in powder cosmetics to the extent that the effect of the present invention is not impaired.

Examples of the other optional components include other powder components (powders that do not fall under components (a), (b), and (c)), an oily component, and an aqueous component.

Examples of the other powder components include a color pigment such as iron oxide; a white pigment such as pigment-grade titanium oxide or pigment-grade zinc oxide; a pearlescent pigment; a spherical powder such as cellulose powder, silicone powder, and boron nitride powder.

These other powder components may or may not be surface-treated.

Examples of the oily component include an oil such as hydrocarbon oil, oil and fat, higher alcohol, wax, hydrogenated oil, ester oil, fatty acid, silicone oil, fluorine-based oil, lanolin derivative, oil-gelling agent, and oily UV absorbing agent.

It should be noted that since introducing a large amount of oily component may induce aggregation of powder, the content of oily component in the cosmetic of the present invention is preferably 10% by mass or less, more preferably 5% by mass or less, further preferably 3% by mass or less, most preferably 2% by mass or less, with respect to the total amount of the cosmetic.

In the powder cosmetic of the present invention, the amount of oil is suppressed, so that the amount of the oily UV absorbing agent is also small (for example, 2% by mass or less). However, since the UV protection ability based on the UV scattering agent is improved (boosted), high SPF can be achieved. Thus, it is also possible to make a powder cosmetic that does not contain an oily UV absorbing agent (so-called “non-chemical” powder cosmetic).

Examples of the aqueous component can include water and water-compatible components, for example, a lower alcohol such as ethanol, a polyalcohol such as glycerin, and a water-soluble polymer.

The cosmetic of the present invention is a powder cosmetic having excellent UV protection ability and good texture, and can be provided in the form of noncompression-molded loose powder or compression-molded pressed powder. The form of products provided is not limited, and the cosmetic of the present invention is particularly suitable for forms such as face powder, foundation, eye shadow, blush, and sunscreen.

### EXAMPLES

Hereinafter, the present invention is described in further detail by way of Examples and the like. However, the following Examples and the like do not limit in any way the scope of the present invention. Unless specified, a content represents % by mass with respect to the total amount of the cosmetic.

Powder cosmetics were prepared according to the conventional methods with the formulations listed in Table 1 below. The cosmetic (10 mg) of each example was applied to a PMMA plate and subjected to a measurement of absorbance to light in the UV wavelength region of 280 to 400 nm, and then the absorbance in the UV wavelength region was integrated.

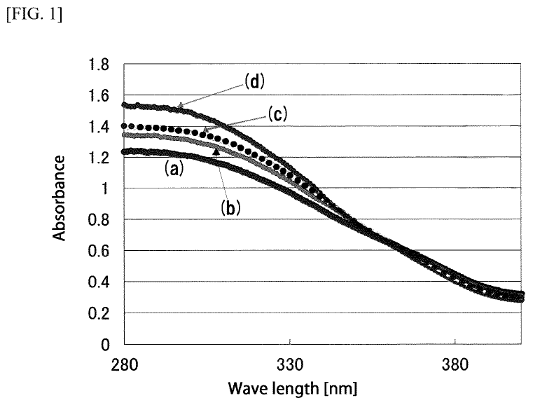

The absorbance curves obtained with the cosmetic of each example are shown in FIG. 1. The absorbance curves shown are of (a) Comparative Example 1, (b) Example 1, (c) Example 2, and (d) Example 3. It can be confirmed that the absorbance to UV increased by containing silica in the cosmetic. The absorbance became higher along with the increase of the content of silica.

The integral value of the absorbance curve obtained from the cosmetic of “Comparative Example 1” was defined as the “reference integral value” and each integral value of the absorbance curves obtained from the cosmetics of Examples 1 to 3 was defined as the “measured integral value”, and the value calculated by the following expression was defined as “boosting ratio” and used as an evaluation index for UV protection ability.

Boosting ratio=(measured integral value/reference integral value)×100

That is, a cosmetic with a “boosting ratio” of more than 100 means that the cosmetic has an improved UV protection ability (i.e., a “boosting effect”) due to the incorporation of silica. It is also recognized that as the value of the boosting ratio becomes higher, the boosting effect becomes greater. The “boosting ratio” for each example is also shown in Table 1.

TABLE 1

| | Comparative | Exam- | Exam- | Exam- |
|---|---|---|---|---|
| | Example 1 | ple 1 | ple 2 | ple 3 |
| Untreated mica | Balance | Balance | Balance | Balance |
| | (70) | (60) | (50) | (40) |
| Spherical silica powder | — | 10 | 20 | 30 |
| (specific surface area: | | | | |
| 600 to 800 m²/g) | | | | |
| Metallic soap-treated | 30 | 30 | 30 | 30 |
| titanium oxide (*1) | | | | |
| Total | 100 | 100 | 100 | 100 |
| UV protection ability | Reference | 106 | 109 | 113 |
| (boosting ratio) | | | | |

(*1) Aluminum stearate-treated titanium oxide (average particle size = 8 nm)

The results (boosting ratio) in Table 1 increased as the silica content increased and were consistent with the results obtained by comparing the absorbance curves in FIG. 1.

The same evaluation as above was then performed for cosmetics where the “untreated mica” in the formulation of Table 1 was replaced with another extender pigment (untreated talc) (Table 2). The formulation and evaluation results (boosting ratio) of each example are shown in Table 2 below. In the cosmetics containing “talc” as an extender pigment (Examples 4 to 6), the “boosting effect” (“boosting ratio”) increased as the silica content increased, as confirmed in the cosmetics containing “mica” (Examples 1 to 3).

TABLE 2

| | Comparative | Exam- | Exam- | Exam- |
|---|---|---|---|---|
| | Example 2 | ple 4 | ple 5 | ple 6 |
| Untreated talc | Balance | Balance | Balance | Balance |
| | (70) | (60) | (50) | (40) |
| Spherical silica powder | — | 10 | 20 | 30 |
| (specific surface area: | | | | |
| 600 to 800 m²/g) | | | | |
| Metallic soap-treated | 30 | 30 | 30 | 30 |
| titanium oxide (*1) | | | | |
| Total | 100 | 100 | 100 | 100 |
| UV protection ability | Reference | 106 | 114 | 150 |
| (boosting ratio) | | | | |

(*1) Aluminum stearate-treated titanium oxide (average particle size = 8 nm)

Here, the same evaluation as above was performed for the cosmetics in which (a) titanium oxide having an average particle size of 10 nm or less in Comparative Example 2 and Example 6 in Table 2 was replaced with titanium oxide having an average particle size of 15 nm (respectively, Reference Example 1 and Reference Example 2). The results are shown in Table 3 below.

In the Reference Examples with the titanium oxide having an average particle size of 15 nm, the “boosting effect” of UV protection ability by incorporating silica was also observed. However, the absolute values of the “boosting ratio” were significantly smaller in comparison with those in the Examples using titanium oxide having an average particle size of 10 nm or less. To illustrate these facts, the boosting ratio of Example 6 calculated using Comparative Example 2 as reference integral value as well as the boosting ratios calculated using Reference Examples 1 and 2 as reference integral value are shown together in Table 3.

TABLE 3

| | Comparative | Reference | Reference | Example |
|---|---|---|---|---|
| | Example 2 | Example 1 | Example 2 | 6 |
| Untreated talc | Balance | Balance | Balance | Balance |
| | (70) | (50) | (40) | (40) |
| Spherical silica | — | — | 30 | 30 |
| powder (specific | | | | |
| surface area: 600 to | | | | |
| 800 m²/g) | | | | |
| Metallic soap-treated | 30 | — | — | 30 |
| titanium oxide (*1) | | | | |
| Metallic soap-treated | — | 30 | 30 | — |
| titanium oxide (*2) | | | | |
| Total | 100 | 100 | 100 | 100 |
| UV protection ability | Reference | — | — | 150 |
| (boosting ratio) | — | Reference | 133 | 165 |
| | — | — | Reference | 124 |

(*1) Aluminum stearate-treated titanium oxide (average particle size = 8 nm)
(*2) Aluminum Stearate-treated titanium oxide (average particle size = 15 nm)

The same evaluation as above was performed for another cosmetic (Example 7) in which the “silica” (porous spherical silica powder) of Example 6 in Table 2 was replaced with a “non-porous silica” having a specific surface area of 10 m²/g or less.

TABLE 4

| | Comparative | Exam- | Exam- |
|---|---|---|---|
| | Example 2 | ple 7 | ple 6 |
| Untreated talc | Balance | Balance | Balance |
| | (70) | (40) | (40) |
| Spherical silica powder | — | 30 | — |
| (specific surface area: ≤10 m²/g) | | | |
| Spherical silica powder | — | — | 30 |
| (specific surface area: 600 to | | | |
| 800 m²/g) | | | |
| Metallic soap-treated titanium | 30 | 30 | 30 |
| oxide (*1) | | | |
| Total | 100 | 100 | 100 |
| UV protection ability (boosting ratio) | Reference | 110 | 150 |

(*1) Aluminum stearate-treated titanium oxide (average particle size = 8 nm)

As shown in Table 4, improvements in UV protection (boosting effect) were observed regardless of whether the “silica” was porous or non-porous. However, the absolute value of the boosting ratio, i.e., the magnitude of the boosting effect, was greater when porous silica was used.

Formulation Examples of the powder cosmetic (loose powder and pressed powder) according to the present invention are described below. In each formulation, good texture without squeakiness and high UV protection ability were obtained.

### Formulation Example 1 (Loose Powder)

TABLE 5

| Component | Content (% by mass) |
|---|---|
| Mica | Balance |
| Metallic soap-treated mica | 10 |
| Boron nitride | 10 |
| Amino acid powder (lauroyl lysine) | 2 |
| Starch powder | 2 |
| Metallic soap (Calcium myristate) | 6 |
| Barium sulfate | 1 |
| Porous silica | 10 |
| Non-porous silica | 15 |
| Metallic soap-treated titanium oxide | 25 |
| Zinc oxide | 7 |
| Preservative | q.s. |
| Antioxidant | q.s. |
| Coloring material | 1.5 |
| Pearly pigment | 0.2 |
| Perfume | q.s. |
| Total | 100 |

### Formulation Example 2 (Loose Powder)

TABLE 6

| Component | Content (% by mass) |
|---|---|
| Mica | Balance |
| Metallic soap-treated mica | 10 |
| Barium sulfate | 1 |
| Porous silica | 30 |
| Non-porous silica | 10 |
| Metallic soap-treated titanium oxide | 25 |
| Zinc oxide | 7 |
| Preservative | q.s. |
| Antioxidant | q.s. |
| Coloring material | 1.5 |
| Pearly pigment | 10 |
| Perfume | q.s. |
| Total | 100 |

### Formulation Example 3 (Pressed Powder)

TABLE 7

| Component | Content (% by mass) |
|---|---|
| Mica | Balance |
| Metallic soap-treated mica | 30 |
| Metallic soap (Calcium myristate) | 5 |
| Porous silica | 10 |
| Non-porous silica | 3 |
| Metallic soap-treated titanium oxide | 15 |
| Zinc oxide | 3 |
| Petrolatum | 1 |
| Sorbitan sesquiisostearate | 1 |
| Triisostearin | 1 |
| Triethylhexanoin | 1 |
| Preservative | q.s. |
| Antioxidant | q.s. |
| Coloring material | 8 |
| Pearly pigment | 8 |
| Perfume | q.s. |
| Total | 100 |

1. A powder cosmetic, comprising:
(a) metal oxide powder consisting of at least one selected from titanium oxide, zinc oxide, and cerium oxide, and having an average particle size of 10 nm or less, in an amount of 5% by mass or more with respect to a total amount of the cosmetic; and
(b) silica.

2. The powder cosmetic according to claim 1, wherein a content of the (a) metal oxide powder is 10% by mass or more with respect to the total amount of the cosmetic.

3. The powder cosmetic according to claim 1, wherein a content of the (b) silica is 10% by mass or more with respect to the total amount of the cosmetic.

4. The powder cosmetic according to claim 1, wherein the (a) metal oxide powder consists of titanium oxide.

5. The powder cosmetic according to claim 1, wherein the (a) metal oxide powder is surface-treated with metallic soap.

6. The powder cosmetic according to claim 5, wherein the metallic soap comprises aluminum stearate.

7. The powder cosmetic according to claim 1, wherein a specific surface area of the (b) silica is 160 m²/g or more.

8. The powder cosmetic according to claim 1, further comprising an oil in an amount of 10% by mass or less with respect to the total amount of the cosmetic.
